(19) United States
(12) Patent Application Publication
(10) Pub. No.: US 2011/0028922 A1
(43) Pub. Date: Feb. 3, 2011

(54) Female External Incontinence Device

(76) Inventors: Dennis M. Kay, Largo, FL (US); David W. Underwood, Tampa, FL (US); Steven J. Babb, Seminole, FL (US)

Correspondence Address: EPSTEIN & GERKEN, 1901 RESEARCH BOULEVARD, SUITE 340, ROCKVILLE, MD, 20850, US

(21) Appl. No.: 12/533,343

(22) Filed: Jul. 31, 2009

(51) Int. Cl. A61F 5/455

(52) U.S. Cl. 604/329

(57) ABSTRACT

A female external incontinence device includes a housing formed of a soft, pliable, membranous, elastomeric material defining a flange for adhesive securement to the intralabial parameatal surface to create a parameatal seal around the urethral meatus, a urine collection channel and an outlet tube and an applicator having a tray component for holding the housing to facilitate securement of the housing to the intralabial parameatal surface.

### BACKGROUND OF THE INVENTION

1. Field of the Invention

The present invention relates to external incontinence devices and, more particularly, to external incontinence devices configured for the human female anatomy.

2. Brief Discussion of the Related Art

Female urinary incontinence is a common medical problem and is, in many cases, attempted to be managed with absorbent diapers or in-dwelling urinary catheters, either urethral or suprapubic. Diapers are associated with a high rate of skin breakdown and decubitis ulcer formation while in-dwelling urinary catheters are a leading cause of urinary tract infections. Accordingly, there has been a great demand for non-invasive external incontinence devices for collecting urine from females without exposing the body to constant urine contact.

There have been many attempts to provide female external incontinence devices as exemplified by U.S. Pat. No. 3,528,423 to Lee, No. 4,484,917 to Blackmon, No. 4,563,183 to Barrodale et al, No. 4,568,339 to Steer, No. 4,681,572 to Tokarz, No. 4,822,347 to MacDougall, No. 4,904,248 to Vaillancourt, No. 4,986,823 to Anderson et al, No. 5,053,027 to Manfredi, No. 5,263,947 to Kay, No. 5,370,637 to Braden, No. 5,830,932 to Kay, No. 6,342,049 to Nichols, No. 6,592,560 to Snyder and, No. 6,699,174 to Bennett. To date, female external incontinence devices have suffered from some or all of the disadvantages of not providing a functional position at anatomical rest such that excreted urine is directed away from the device, not effectively conforming to the parameatal surfaces in a position of anatomical rest such that excretal urine is directed away from the device, not providing an occlusive seal around the urethral meatus, not functionally managing urine output during use of the device in normal daily activities, requiring constant manual pressure to direct urine into a collection receptacle, being difficult to accurately place relative to the urethral meatus, requiring deployment by medical personnel, not being universal in nature relative to anatomical variations, failing to direct urine away from the body, requiring shaving and/or lateral distraction of the labia and thighs and/or utilizing firm or rigid structures or structures not matching the physical profile of the target anatomy with the wearer's body in normal positions, with labia, legs and thighs together in the midline.

The most problematic disadvantages of prior art female external incontinence devices is the discomfort associated with the relatively rigid or hard (high durometer) urine collection housings of the devices, particularly during normal daily activities, and the need for apparatus to press the housings against tissue surrounding the urethral meatus.

### SUMMARY OF THE INVENTION

In one aspect of the present invention, a female external incontinence device includes a housing having a structure for adhesive attachment to tissue in the vaginal opening (introitus), in particular, the labia minora (labium minus), an intravaginal component extending from the flange at an angle to be inserted in the vagina to anchor the housing without occluding the vagina, a urine collection channel depending from the adhesive attachment structure and an outlet tube communicating with the urine collection channel, the adhesive attachment structure having an elongated opening therein to facilitate alignment of the housing with the urethral meatus regardless of where the urethral meatus presents while maintaining a parameatal seal around the urethral meatus.

In another aspect, a female external incontinence device includes a housing made of an elastomer forming, as a single part, a flange for attachment to tissue in the vaginal opening (introitus), in particular, the labia minora (labium minus), a intravaginal component extending from the flange at an angle to be inserted in the vagina, a urine collection channel depending from the flange and an outlet tube communicating with the urine collection channel, and an applicator having retainer and tray components disposed above and below the housing to sandwich the flange and the intravaginal component therebetween to facilitate deployment of the housing in the vaginal introitus.

A further aspect of the present invention is to provide a female external incontinence device including an elastomeric housing forming a flange, a urine collection channel communicating with an opening in the flange and an intravaginal component extending angularly from the flange to be received in the vagina, the housing having a shape that prevents buckling at a curved or angled portion between the flange and the intravaginal component when the housing is deformed thereby maintaining a parameatal seal around the urethral meatus.

Another aspect of the present invention is to improve comfort in a female external incontinence device by utilizing a housing made of an elastomeric material that is sufficiently soft and pliable to conform to tissue at the application site in normal positions of anatomical rest without causing injury or discomfort during wear while permitting urine flow therethrough.

In a further aspect, the present invention utilizes protuberances on the inner surfaces of walls of a housing of a female external incontinence device to permit flow of urine therethrough when the housing is collapsed along a midline in compliance to female anatomical structure such that the housing can be soft and pliant without occluding urine flow.

In another aspect of the present invention, the female external incontinence device is conformed to allow all the tissues of the introitus, fourchette, perineum and labia to return and remain in the normal “at rest” position with no distraction of the labia and with the labia minora flaccid with the medical aspect of the labia in direct approximation to each other and the thighs adducted to the midline or “closed” position of anatomical rest whether the wearer is seated, standing, lying supine, prone or walking.

A further aspect of the present invention is the conformation of a parameatal housing and a parameatal barrier body combined such that the central meatal opening of the barrier body can conform to the plane of the perineum and the plane of the anterior vaginal wall. The plane of the perineum and the plane of the anterior vaginal wall in female anatomy presenting in an at rest position meet at an angle generally ranging from 140° to 80°. The conformation of the parameatal barrier body and housing combine to provide a mating surface that is flexible and a parameatal opening that can flexibly mate with the perineum and the anterior vaginal wall throughout an angle varying from approximately 80° to 140° to optimize functionality of the female external incontinence device since the location of the urinary meatus in normal female anatomy can vary from the anterior, just below the clitoris, to the inner aspect of the vaginal verge, contiguous with the anterior vaginal wall.

Some of the features of the present invention providing advantageous use thereof include a urine collection chamber configured to enter the introitus and ensure collection of urine from the urethral meatus regardless of the position of the urethral meatus from just below the clitoris to within the introitus, a housing made of a soft, pliable, elastomeric material to be adhesively secured and worn in comfort to provide an occlusive parameatal urethral seal without requiring rigid or firm structures, alignment for proper placement or deployment of the external incontinence device facilitated even in those cases where the exact location of the urethral meatus cannot be visualized, a housing having midline collapsibility while permitting urine flow therethrough such that the anatomy does not remain distended during use, and a housing applied to the vaginal region in a manner to smooth out the rugae folds and creases of tissue to provide a smooth, relatively flat, sealing surface for receiving the parameatal barrier formed on the flange of the housing thereby optimizing a flat, wrinkle-free parameatal seal to produce effective, leak-free urine evacuation.

Other aspects and advantages of the present invention will become apparent from the following description of the exemplary embodiments taken in conjunction with the accompanying drawings, wherein like parts in each of the several figures are identified by the same reference characters.

### BRIEF DESCRIPTION OF THE DRAWINGS

FIGS. 1 and 2 are opposite perspective views of a housing of a female external incontinence device according to the present invention.

FIGS. 3 and 4 are views of the housing of a female external incontinence device according to the present invention showing alternative protuberance configurations to facilitate urine flow.

FIGS. 5 and 6 are side and perspective exploded views, respectively, illustrating the relationship of the housing with an applicator of the female external incontinence device of the present invention.

FIG. 7 is a perspective view of a female external incontinence device according to the present invention with the housing held in the applicator.

FIG. 8 is a perspective view of the female external incontinence device of the present invention with the retainer component of the applicator being removed and removal of the release film initiated.

FIGS. 9, 10 and 11 are side views of the female external incontinence device of the present invention with the female anatomy in cross-section, illustrating deployment of the housing, withdrawal of the applicator and use of a bag for collecting urine, respectively.

FIG. 12 is a view of the vaginal vulva with the labia minora distracted and the female external incontinence device housing of the present invention secured in place on the intralabial parameatal surface with the urine collection channel, bulging formation and outlet tube not shown.

FIG. 13 is a front view of the housing of the female external incontinence device of the present invention secured in the vaginal introitus with the labia minora distracted and the housing in an anatomically conformed condition.

FIG. 14 is a perspective view of the housing of the female external incontinence device of the present invention in an anatomically conformed condition.

FIG. 15 is an exploded perspective view of a modification of the applicator of the female external incontinence device of the present invention with the housing in phantom.

FIG. 16 is a perspective view of the applicator of FIG. 15 in use.

FIG. 17 is an exploded perspective view of another modification of the applicator of a female external incontinence device of the present invention.

### DETAILED DESCRIPTION OF EXEMPLARY EMBODIMENTS OF THE INVENTION

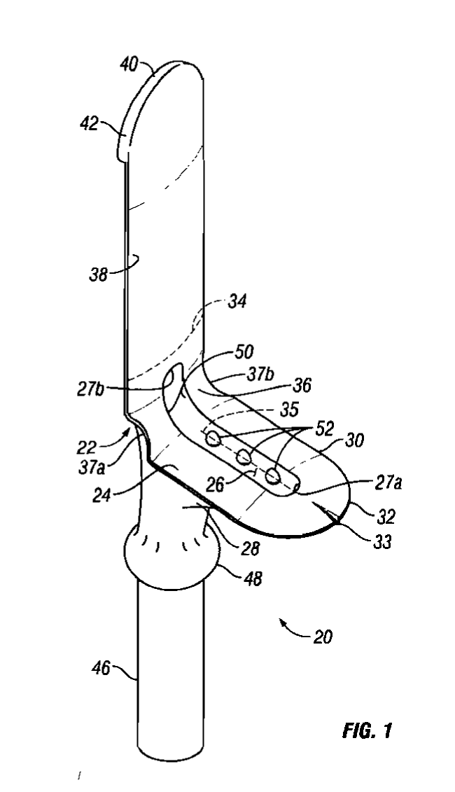

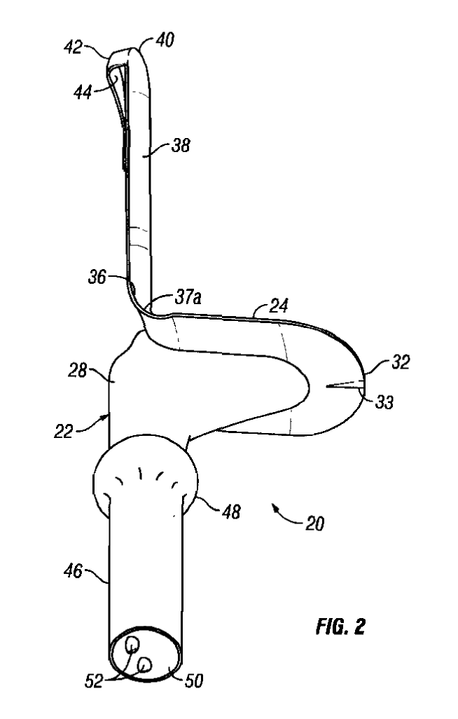

As shown in FIGS. 1 and 2, a female external incontinence device 20 according to the present invention includes a housing 22 having a flange 24 defining an elongate opening 26 therethrough communicating with a urine collection channel 28 depending from the flange. The opening 26 has curved, opposed proximal and distal ends 27a and 27b, respectively. The flange 24 forms a structure for adhesive attachment to tissue in the introitus and can have a margin 30 with an arcuate or curved anterior end 32 or, depending on the manner in which the housing 22 is formed, the flange 24 can have a minimal lateral dimension as compared to the longitudinal dimension extending from the end 32 to a location 34 posterior of the end of opening 26 shown by a dotted line. The anterior portion of flange 24 has a V-shape in an end-wise view looking posteriorly along the flange to define a fold-type line 33 aligned with a midline of housing 22 shown in dotted lines at 35 such that the housing is biased to collapse along the midline. The flange 24 has a curved portion 36 to produce an angle of approximately 105° between the plane of the flange 24 and the plane of an intravaginal component 38 which, as shown in the embodiment shown in FIG. 1, is formed by a tongue-shaped anchor extending from the flange at the approximately 105° angle and configured to be received in the vagina to act as an anchoring member. The side edges of flange 24 adjacent curved portion 36 have opposing arcuate recesses 37a and 37b on opposing sides of the flange. The flange and the intravaginal component are curved along a direction transverse to midline 35 as represented by the curvature shown by dotted line 34. That is, the tissue engaging surfaces of the housing have a lateral curvature relative to the longitudinal dimension of the housing corresponding to midline 35 to provide a domed-like shape. The intravaginal component 38 terminates at a rounded distal end 40 carrying a pocket 42 formed on an outer side thereof and defining an opening 44 formed between layers of the material from which housing 22 is made. The urine collection channel 28 communicates with an outlet tube 46 through a bulging formation 48 which facilitates movement of the outlet tube and any tubing connected therewith to avoid kinking. The housing 22 is molded of a suitable elastomeric material to be soft, membranous, flexible and pliable and, thus, comfortable during use, and the parts of the housing have thin walls so as to be membranous. The bulging formation 48 can have any shape to avoid kinking, for example a rounded spherical type shape (as shown), a bellows shape or other shapes, and has a diametrical dimension greater than the diametrical dimension of the outlet tube.

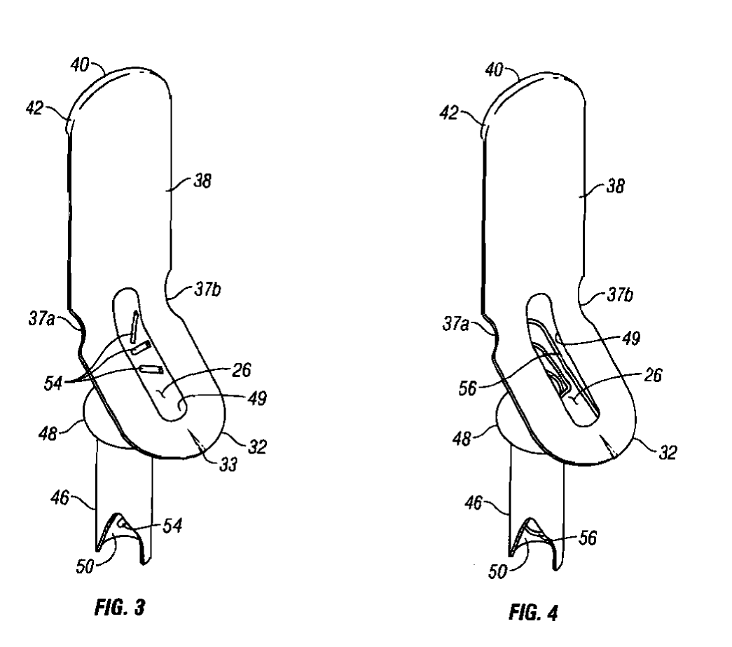

The inner wall surfaces 50 of the housing 22, particularly in the urine collection channel 28 and the outlet tube 46, have protuberances 52 extending inwardly therefrom to allow urine to pass through the urine collection channel 28 and outlet tube 46 when the housing 22 is collapsed along its midline in use. The protuberances 52 are shown as being semi-spherical or bead-like in FIGS. 1 and 2, and are arranged in a pattern such that a fluid path extends through the urine collection chamber and the outlet tube even when the housing is collapsed. Alternative protuberance arrangements and shapes are shown in FIG. 3 as rods 54 in a lattice or crossing pattern to cross each other when the housing is collapsed arid, as shown in FIG. 4, rods 56 having a helical arrangement extending from the outlet tube 46 and expanding into the urine collection channel 28 to end where the urine collection channel meets the flange 24 at the opening 26. Alternatively, a significant domed ridge or pair of ridges can run the length of the urine collection channel and outlet tube to create a similar spacing effect between the inner wall surfaces 50 in the event the walls are collapsed in use.

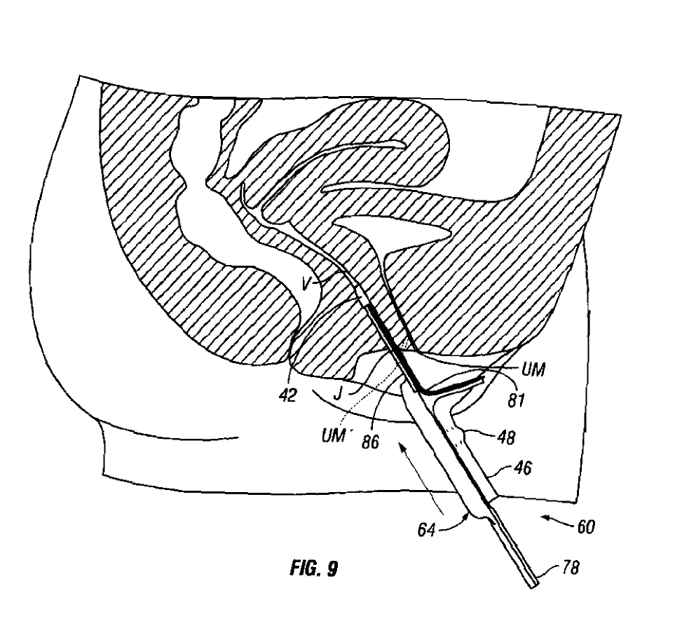

The intravaginal component 38 and the flange 24 have inner surfaces covered with an adhesive layer 58 as best shown in FIG. 8; and, prior to use, the surface of the adhesive layer are covered with a release film as shown at 59 in FIG. 8. The adhesive layer can be formed of any medically acceptable adhesive, such as a hydrocolloid which is elastic in nature. The membranous width of margin 30 around opening 26 can vary in structure dependent on the method of molding housing 22. That is, flange 24 with margin 30 can be molded unitarily with housing 22; or, if molding leaves only a small rim surrounding opening 26, margin 30 can be formed by attaching the layer of adhesive 58 to the rim. The adhesive layer on the flange extends entirely around opening 26 such that a parameatal seal is formed around the urethral meatus which is positioned within the perimeter of the opening in use of the female external incontinence device. The adhesive layer 58 on intravaginal component 38 extends along the length thereof to maximize securement in the vagina but may extend only partially along the distal end of the intravaginal component as long as adequate securement in the vagina is achieved and is covered by a release film, backing or liner 59.

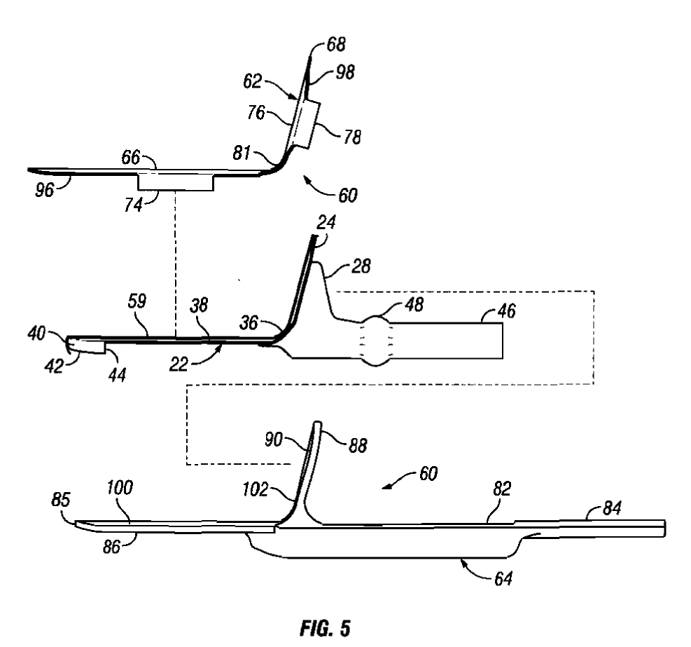

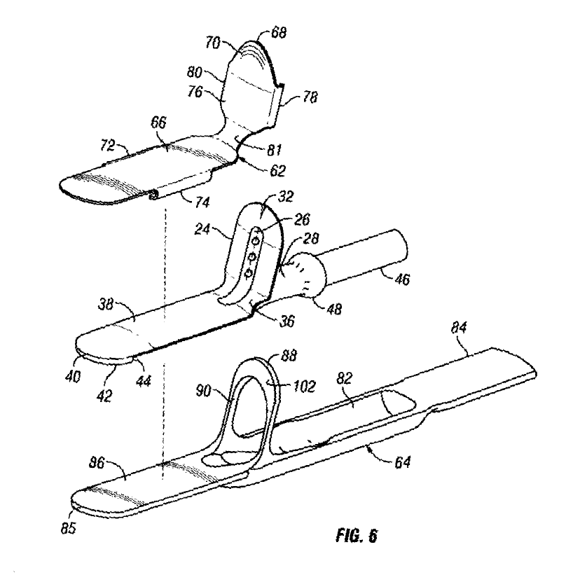

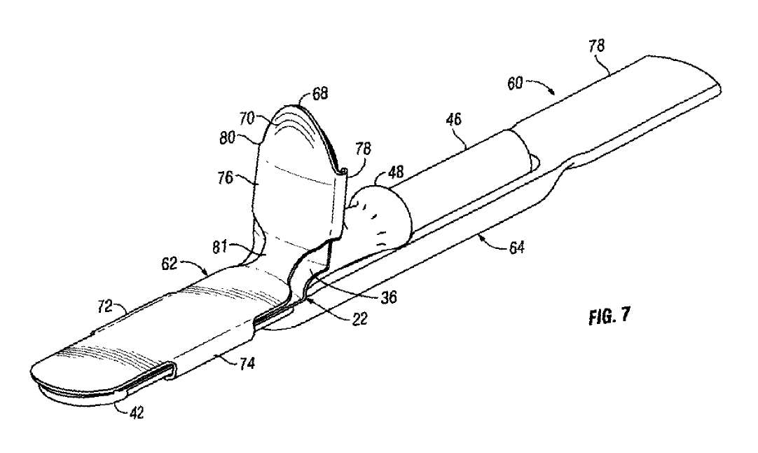

The female external incontinence device 20 includes an applicator 60 as shown in FIGS. 5, 6, 7 and 8, the applicator including a retainer component 62 and a tray component 64. The retainer component 62 has a configuration conforming with the configuration of the flange 24 and intravaginal component 38 with the exception that the retainer component has a length to form a pull tab 68 for extending beyond a proximal end of flange 24 when the retainer component 62 is clipped to the tray component 64. As shown in FIG. 6, the pull tab 68 carries ridges 70 to facilitate grasping thereof and is attached to the release film 59 via a bead of glue 92. Depending from opposing lateral sides of a portion 66 of retainer component 62 configured to overlie intravaginal component 38 are flexible clips 72 and 74 and, similarly, extending from opposing lateral sides of a flange covering portion 76 of retainer component 62 configured to conform to flange 24 are flexible clips 78 and 80. The flange covering portion 76 has a reduced width neck 81 connected with intravaginal component covering portion 66 and curved to correspond to, and overlie, curved portion 36 of flange 24. The tray component 64 has a concavity 82 in a grip portion 84 and has an opposing, rounded, distal end 85 of a intravaginal component deployment portion 86 configured to be received in pocket 42 at the distal end of intravaginal component 38 of housing 22. The concavity 82 has dimensions to conform to the dimensions of urine collection chamber 28, bulging formation 48 and outlet tube 46. The tray component 64 has a flange-supporting and recess-containing member 88 extending therefrom with a recess/opening 90 therein to permit passage therethrough of the urine collection channel, the pivot bulge and the outlet tube.

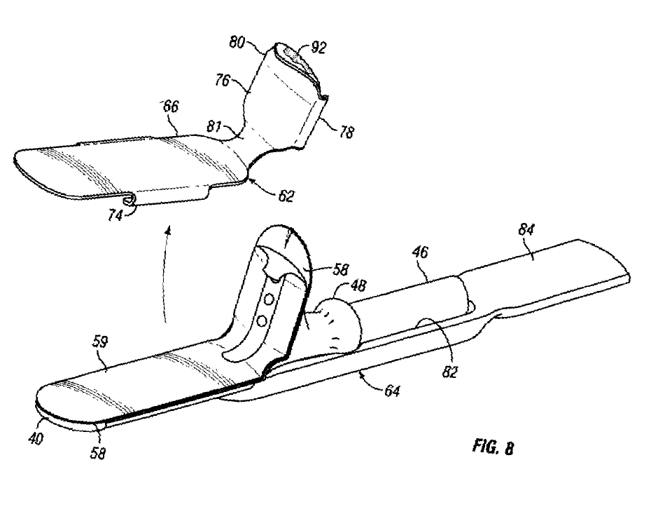

In use, female external incontinence device 20 is supplied with the housing 22 disposed in the applicator 60 as shown in FIG. 7. The urine collection channel 28, the bulging formation 48 and the outlet tube 46 are received in the concavity 82 in the tray component 64 and pass through recess 90 which is an opening in the embodiment illustrated. If recess 90 has a closed end, the urine collection chamber 28, the bulging formation 48 and the outlet tube 46 will be received therein, The retainer component 62 is detachably connected with the tray component 64 via opposing clips 72 and 74 flexibly engaging the intravaginal component deployment portion 86 of tray component 64 and opposing clips 78 and 80 flexibly engaging the recess containing member 88. Accordingly, retainer component 62 and tray component 64 are disposed on opposite sides of housing 22 to detachably sandwich the housing 22 within the applicator with the adhesive layer 58 and release film 59 between the housing and the retainer component. The release film can be coupled to the retainer component in any suitable manner, such as by the spot or bead of glue 92, carried adjacent pull tab 68.

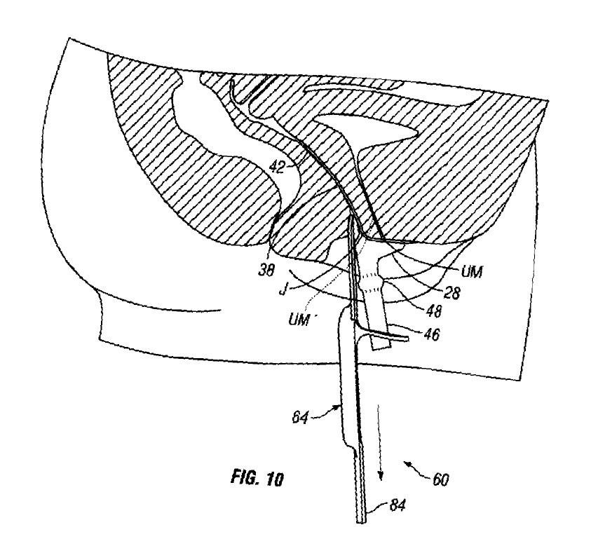

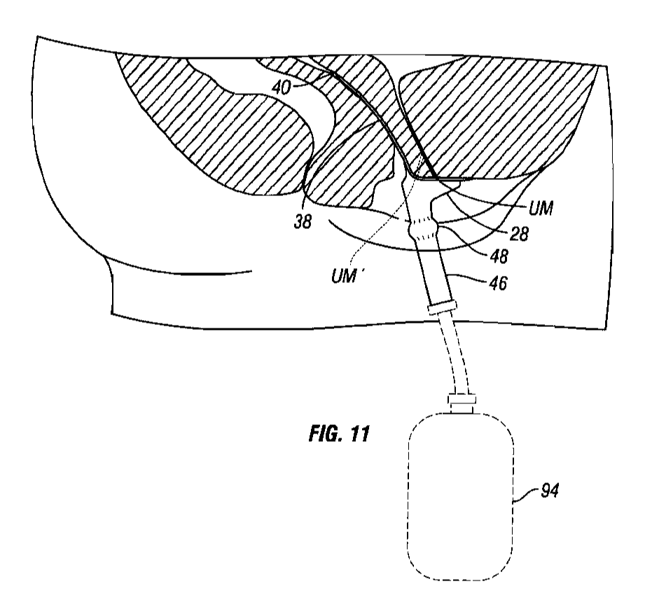

To position the housing 22 in the vaginal introitus, the grip portion of the applicator 60 is held in the hand, and the tongue deployment portion 86 of the applicator is inserted in the vagina as shown in FIG. 9, the intravaginal component 38 of the housing 22 being deployed in the vagina due to the distal end 85 being received in the opening 44 of the pocket 42. The retainer component of the applicator is preferably removed after placement of the housing; however, in some cases, the retainer component can be removed prior to insertion of the intravaginal component 38 in the vagina. In FIGS. 9, 10 and 11, the urethral meatus UM is depicted in solid lines in its normal presentation. However, as noted above, the urethral meatus can present at various locations in different females, a more posterior location UM' being shown in dotted lines. Upon full insertion, the flange curved portion 36 engages the curved junction of the intralabial portion of the parameatal surface with the anterior vaginal wall such that the margin 30 forms a seal around the urethral meatus regardless of the anatomical location of the meatus. Once the female external incontinence device is fully inserted, the applicator is withdrawn, noting that the release film 59 is pulled from the adhesive layer 58 as the retainer component is removed. Removal of the retainer component of the applicator is facilitated by the pull tab 68 extending beyond the end of the flange covering portion 76. Once the release film is removed, the adhesive layer secures the intravaginal component to the anterior vaginal wall, and the flange is secured in a position to provide a seal around the urethral meatus via the adhesive layer. With the housing adhesively secured in place, the applicator is withdrawn as depicted in FIG. 10. FIG. 11 illustrates the outlet tube of the housing being coupled with a collection bag or other reservoir.

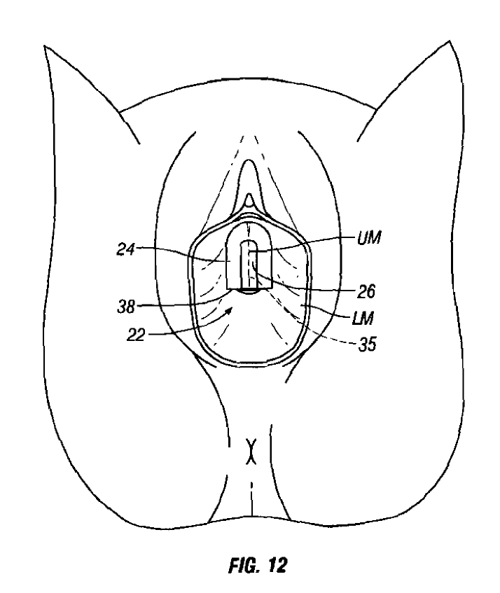

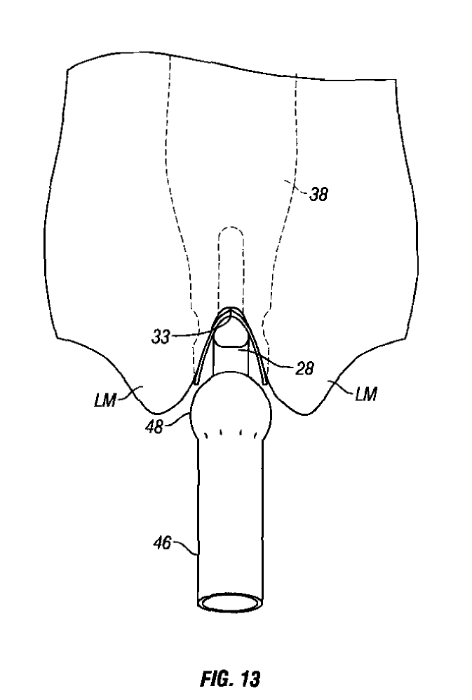

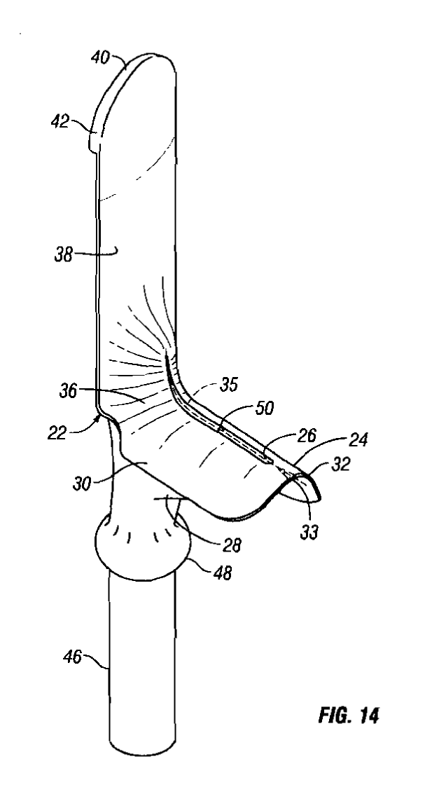

As shown in FIG. 12, with the housing 22 adhesively secured in the vaginal vault, the flange 24 produces an occlusive parameatal seal around the urethral meatus UM via the adhesive securement to the intralabial parameatal surface within the labia minors LM, the elongated shape of the opening 26 permitting the housing to be used even with variations of the anatomical location of the urethral meatus. The view shown in FIG. 12 is with the housing in an open position and without the urine collection chamber, the bulging formation and the outlet tube, and it is noted that the flange folds along midline 35 in use as shown in FIGS. 13 and 14.

As previously explained, the flange flexes toward the urine collection channel in the rest position. That is, the housing will collapse during use to have the position shown in FIGS. 13 and 14 with the membranous walls of the flange flexed toward the urine collection channel 28 and outlet tube 46. Accordingly, the housing 22 is comfortable in use and permits normal daily activities with the conformation of the housing fitting the anatomical position of rest without occlusion of urine flow through the urine collection channel and the outer tube due to the use of protuberances on the inner surfaces of the housing. The initial lateral curvature of the margins of the flange permit the housing to fold along a longitudinal (sagittal) midline of the labial portion of the parameatal area which will be aligned with the midline 35.

An example of a female external incontinence device constructed in accordance with the present invention follows. The housing 22 is made of a thin, soft, pliable, membranous elastomeric material. The housing membrane can have a thickness of from 0.002 to 0.009 inches and the protuberances can extend from the inner surfaces of the membrane along the urine collection channel and adjacent the flange and in the outlet tube to have a thickness, inclusive of the membrane, of 0.017 inches. The elastomeric material has a flexural modulus below 30,000 psi, elongation greater than 200%, a durometer at or below Shore A 30, a static coefficient of friction below 0.3 and a kinetic coefficient of friction below 0.1. Elastomeric materials that can be used to form the housing include polyurethane, nitrile and polyisoprine as well as embodiments of polyvinylchloride when the housing is injection molded. The housing is made as a membrane of the elastomeric material which is of minimal thickness. The intravaginal component can have a length from the point 34 to the end 40 of the pocket 42 of 5.0 cm, the urine collection channel can be offset downward 5.0 mm below the plane of the intravaginal component and can be 2.5 cm in height from the bottom to the top of the parameatal adhesion surface, i.e. the flange 24, the bulging formation 48 can bulge approximately 2.0 mm above and below the outlet tube surface and have any desired shape but the sides of the outlet tube exhibit no bulge around each side of the device, the width of the urine collection channel can be 5.0 mm throughout the channel until the channel meets the bulging formation, distal of the pivot bulge the outlet tube assumes a 9.0 mm inner diameter dimension, the flange/margin can be 7.0 mm from the edge of the urine collection channel opening 26 to the edge of the flange/margin on the top left and right sides as viewed from the distal end, the angle or curvature between the intravaginal component and the flange will complete a 105° curve to enter the introitus, the urine collection channel can terminate 1.0 cm past termination of the curved portion of the flange, the total length of the housing can be 11.5 cm. Of course, the dimensions can change to allow the female external incontinence device to be used by females not fitting into the 75% population.

Due to the design of the flange and the urine chamber opening, the housing will fit various sized vaginal vaults with varying urethral meatus presentations. The housing contact surface of the retainer component of the applicator is curved from lateral edge to lateral edge, as shown in FIG. 5, such that intravaginal component covering portion 66 and flange covering portion 76 have housing contact surfaces 96 and 98, respectively, with curvatures conforming to the convex surface 100 of intravaginal component deployment portion 86 and the convex inner surface 102 of recess containing member 88, respectively, and also with the lateral curvature of housing 22. The flange 24 has similar lateral curvature in the rest position such that sandwiching of the flange between the retainer and tray components of the applicator maintains the desired application shape of the flange prior to deployment. The shape of the housing and curvature of the flange aids midline collapse of the housing and optimum conformation to the intralabial parameatal surface and to the intraoital transitional anatomy. The 105° curve accommodates the transitional tissue or junction between the intralabial parameatal surface and the anterior vaginal wall as the flange is adhesively attached to all of these surfaces in a contiguous manner.

The applicator is used to ship, store and deploy the housing and can be formed from a plastic such as polystyrene, polyvinylchloride or polypropylene with the plastic slick to the touch and having “self-lubricating” characteristics for vaginal insertion in a manner similar to plastic tampon applicators. The plastic allows the elastomeric material of the housing to slide easily across the applicator surface to aid withdrawal of the applicator once the housing is in confirmation with the anatomy. The applicator essentially forms a sandwich of two plastic components to hold the thin, soft, highly flexible and pliable housing in position therebetween from manufacture to deployment. The retainer component of the applicator holds the housing in conformation with the tray component of the applicator and provides a means for removing the release film which can be attached at the tab by an adhesive bead. For optimal effectiveness, the release film is removed in a manner so that the most proximal portion of the adhesive surface 58 is the first portion of the adhesive surface to be exposed to the application surfaces. The retainer component also holds the housing in conformation with the tray component of the applicator by lightly snapping thereto via the clips 72, 74, 78 and 80 to hold the housing in proper position for shipping and deployment. The release film removal is accomplished by pulling the retainer component of the applicator away from the tray component by grasping the tab and pulling the release film away from the housing with the clips deforming to allow detachment of the retainer component from the tray component. The release film can be a type of silconized polyurethane film similar to the Argotec urethane film 27303. The tray component of the applicator acts as a support channel for the housing and provides an insertion function due to the insertion end being received in the intravaginal component pocket, it being noted that intravaginal component 38 is flaccid and flexible due to its membranous nature and that the intravaginal component deployment portion imparts rigidity to facilitate deployment of the intravaginal component against the anterior vaginal wall and then proceeding in a contiguous moving front of application to the intralabial portion proximal the parameatal surface. When the applicator is removed, the thin soft, flexible, pliable nature of the entire housing provides protection of the application site from crush injury, pressure injury and/or ischemia. The applicator facilitates adhesion through equal application of force during deployment and withdrawal.

As shown in FIGS. 13 and 14, the lateral curvature and narrowed width (relative to the width of the intravaginal component) of the flange margins toward the urine collection channel prevents buckling of the flange adjacent the curved portion 36 when the housing is in use. Rather, the flange lateral curvature causes the curved portion 36 to either remain in alignment with the remainder of the flange or to bulge slightly. Accordingly, the seal of the flange with the intralabial surface around the urethral meatus remains intact when the housing assumes conformation with the anatomical position of rest since the flange margin convexity enhances the seal formation within the concavity formed by the intralabial parameatal surface. That is, the housing 22 has a longitudinal dimension along midline 35 and a transverse lateral dimension such that the flange 24 has at least a partial lateral curvature about the longitudinal dimension in a resting state.

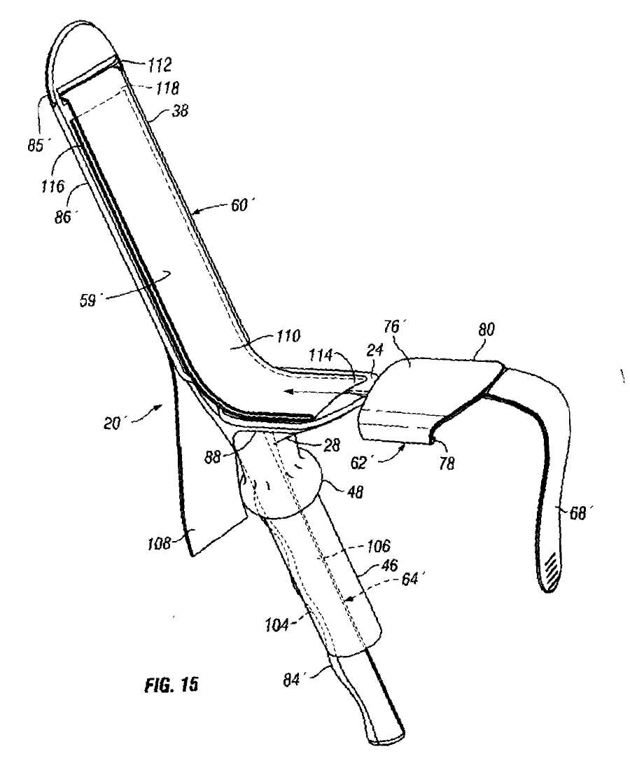

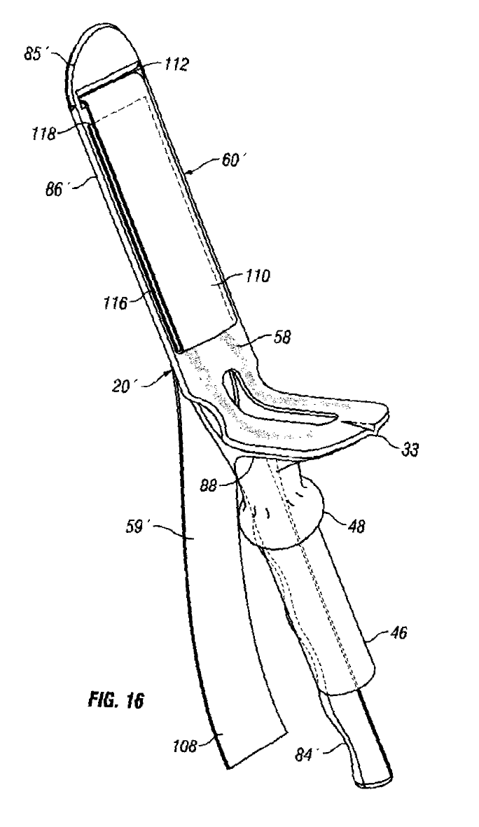

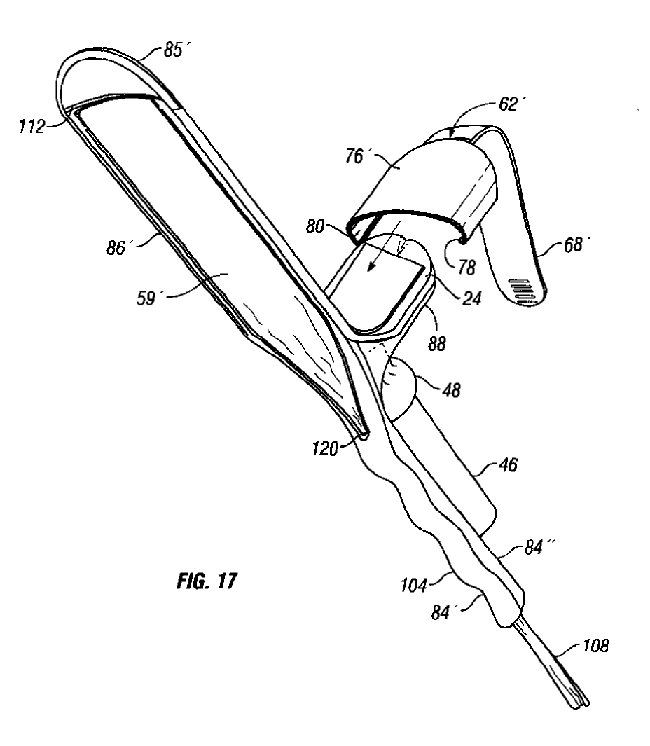

A modification 20″ of the female external incontinence device of the present invention is shown in FIGS. 15 and 16 with parts identical to parts of female external incontinence device 20 given identical reference numbers and parts similar to parts of female external incontinence device 20 given the same reference numbers with primes added. The primary difference between the female external incontinence devices 20 and 20′ is the design of the applicator 60′ relating to holding of the housing 22 during shipping, storage, handling and deployment and removal of release film 59′ prior to adhesive securement of the housing on the body.

As shown in FIGS. 15 and 16, applicator 60′ includes a retainer component 62′ and a tray component 64′. Retainer component 62′ carries flexible clips 78 and 80 on opposing lateral sides of a flange covering portion 76′ to engage the sides of flange-supporting, recess-containing member 88 of tray component 62′. A pull tab 68′ extends from flange covering portion 76′ allowing the retainer component to be disengaged (unclipped) from the tray component 64′ by grasping the pull tab.

The tray component 64′ has a grip portion 84′ with a bottom, scalloped, finger grip surface 104 and an upper surface 106 for supporting urine collection channel 28, bulging formation 48 and outlet tube 46. The release film 59′ is folded on itself to have a U-shape with a free end 108 extending along the grip portion 84′ of tray component 64′, a length 110 extending along the outer side of the intravaginal component 38 and the flange 24 after passing through a slot-like opening 112 in a distal end 85′ of intravaginal component deployment portion 86′, and the release film 59 is folded on itself at 114 adjacent the anterior end of the flange-supporting, recess-containing member 88 to form a length 116 leading to a secured end 118 at the distal end 85′ adjacent opening 112. Accordingly, the release film is folded on itself at opening 112 and at fold 114 to have a U-shape. The length 116 of the release film covers the layer of adhesive 58 on intravaginal component 38 and flange 24 prior to deployment of the female external incontinence device on the body.

As shown in FIG. 16, after removal of the retainer component 62′ prior to deployment, the free end 108 of the release film 59′ is pulled anteriorly such that the release film moves through the opening 112 to expose the adhesive layer 58 on the housing 22 to facilitate adhesive securement of the housing to the intralabial parameatal surface to create a parameatal seal around the urethral meatus.

Another modification is shown in FIG. 17 which differs from the modification shown in FIGS. 15 and 16 in that the length of release film 110 passes through a passage 120 in the grip portion 84″ such that the release liner does not hang loosely.

In accordance with a method of use of the female external incontinence device 20 according to the present invention, a patient is in a lithotomy position or recumbent on her back, in gynecologic stirrups with her knees flexed, her thighs abducted and her feet flat on the bed in the area of the buttocks. In the first step, the applicator is initially inserted with the split or space between the retainer component and the tray component parallel to the split between the labia. Initial insertion of the applicator will spread the labia. The applicator is then rotated 90° to bring the retainer component in contact with the anterior vaginal wall in such a way as to spread the rugae that are found present in the introitus. The rugae run in a horizontal, somewhat circumferential plane about the anterior vaginal wall, and the retainer component is pressed up and forward as it is delivered into the introitus in order to spread the rugae to flatten them. The tray component is held by the grip portion with a modest amount of upward and forward pressure to keep the rugae spread and flattened while the retainer component and/or the release film is removed to expose the adhesive to a largely flat and wrinkle-free surface which, when allowed to go to rest, will let the rugae return. Once the adhesive is exposed by removing the release film to expose the adhesive with a hand supporting the housing so the housing is not inadvertently withdrawn, the tray component is withdrawn without rotation and taking care not to dislodge the housing.

The use of an elastomeric material to form the housing in cooperation with an elastic layer of adhesive, (e.g. hydrocolloid) provides elastic compression and permits comfortable use of the female external incontinence device while also providing an effective seal around the urethral meatus. The unitary formation of the housing and the intravaginal component of a soft, pliable, membranous, elastomeric material provides the advantages of ease of manufacture and positioning on the body as well as comfort in use; however, the intravaginal component can take other forms, such as a pessary or balloon structure, coupled with the housing.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. A female external incontinence device comprising
a housing having a flange for attachment to tissue in the vaginal opening, a urine collection channel depending from said flange and an outlet tube communicating with said urine collection channel, said flange having a configuration to define a margin surrounding an elongated opening for alignment with the urethral meatus;
an intravaginal component extending from said flange at an angle to be inserted in the vagina; and
an adhesive layer carried on said flange margin for securing said housing in a sealing manner around the urethral meatus.

2. A female external incontinence device as recited in claim 1 wherein said outlet tube has a diametrical dimension and said housing includes a bulging formation disposed between, and communicating with, said urine collection channel and said outlet tube, said bulging formation having a diametrical dimension greater than the diametrical dimension of said outlet tube.

3. A female external incontinence device as recited in claim 1 wherein said tongue is formed of a flexible membrane, is connected with said flange and terminates at a distal end forming a pocket between layers of said flexible membrane to facilitate deployment of said housing and said intravaginal component.

4. A female external incontinence device as recited in claim 3 wherein said pocket is formed on an outer side of said intravaginal component and an adhesive layer is carried on an inner side of said intravaginal component opposite said outer side.

5. A female external incontinence device as recited in claim 1 wherein said housing is formed of a single piece of soft, membranous, flexible, pliable elastomeric material whereby said flange, said urine collection channel and said outlet tube are unitarily formed as a single part.

6. A female external incontinence device as recited in claim 5 wherein said soft, membranous, flexible, pliable elastomeric material has a durometer of Shore A 30 or less.

7. A female external incontinence device as recited in claim 6 wherein said housing has a longitudinal dimension and a lateral dimension and said flange has at least a partial lateral curvature about the longitudinal dimension in a resting state.

8. A female external incontinence device as recited in claim 7 wherein said curvature of said flange is formed by opposing lateral sides of said margin curving toward said urine collection channel.

9. A female external incontinence device as recited in claim 1 wherein said flange of said housing has a longitudinal dimension defining a midline for said housing and an anterior portion with a shape defining a fold line aligned with said midline whereby said housing is biased to collapse along said midline.

10. A female external incontinence device as recited in claim 9 wherein said urine collection channel has an inner surface adjacent said opening in said flange and protuberances extending from said inner surface to enhance urine flow.

11. A female external incontinence device as recited in claim 1 wherein said urine collection channel and said outlet tube have inner surfaces and protuberances extending from said inner surfaces to enhance urine flow.

12. A female external incontinence device as recited in claim 1 wherein said housing and said intravaginal component are formed of a single piece of soft, membranous, flexible, pliable elastomeric material whereby said flange, said urine collection channel, said outlet tube and said intravaginal component are unitarily formed as a single part.

13. A female external incontinence device comprising
a unitary housing made of an elastomer forming, as a single part, a flange for attachment to parameatal tissue in the vaginal opening,
a urine collection channel depending from said flange and an outlet tube communicating with said urine collection channel;
an intravaginal component extending from said flange at an angle to be inserted in the vagina; and
an applicator including a tray component supporting said housing and said intravaginal component and disposed under said flange.

14. A female external incontinence device as recited in claim 13 wherein said intravaginal component terminates at a distal end forming a pocket and said tray component of said applicator terminates posteriorly at a distal end received in said pocket to facilitate deployment of said housing.

15. A female external incontinence device as recited in claim 13 wherein said tray component includes a concavity receiving said urine collection channel and said outlet tube.

16. A female external incontinence device as recited in claim 13 wherein said applicator includes a retainer component disposed over said flange and detachably coupled with said tray component to sandwich said housing therebetween.

17. A female external incontinence device as recited in claim 16 wherein said flange has an outer surface carrying an adhesive layer for adhesive attachment of said housing to the intralabial parameatal surface surrounding the urethral meatus and further comprising a release film covering said adhesive layer of said flange and coupled with said retainer component of said applicator so as to be removed from said flange when said retainer component of said applicator is withdrawn.

18. A female external incontinence device as recited in claim 13 wherein said flange has an outer surface carrying an adhesive layer for adhesive attachment of said housing to the intralabial parameatal surface surrounding the urethral meatus and further comprising a release film covering said adhesive layer of said flange and removable for adhesive deployment of said housing.

19. A female external incontinence device as recited in claim 18 wherein said release film cooperates with said tray component to retain said housing on said tray component.

20. A female external incontinence device as recited in claim 13 wherein said flange has an outer surface carrying an adhesive layer for adhesive attachment of said housing to the intralabial parameatal surface surrounding the urethral meatus and further comprising a release film having a U-shape with a free end extending along said tray component such that said release film can be removed to expose said adhesive layer by pulling said free end.

21. A female external incontinence device as recited in claim 13 wherein said flange has an outer surface carrying an adhesive layer for adhesive attachment of said housing to the intralabial parameatal surface surrounding the urethral meatus and said intravaginal component has an outer surface carrying an adhesive layer for adhesive attachment of said housing to the anterior wall of the vagina and further comprising a release film covering said outer surface of said flange and said outer surface of said intravaginal component and coupled with said retainer component of said applicator so as to be removed from said flange and said intravaginal component when said retainer component of said applicator is withdrawn.

22. A female external incontinence device comprising
a housing made of soft, pliable, elastomeric, membranous material configured to define a flange having an opening therein with said opening having a longitudinal dimension greater than a width dimension of said opening to provide an elongated shape for surrounding the urethral meatus;
a urine collection channel communicating with said opening to collect urine exiting from the urethra;
an outlet tube communicating with said urine collection channel; and
an elastic layer of adhesive carried by said flange and surrounding said opening to contact tissue adjacent the urethral meatus to provide a urethral parameatal seal,
said housing and said layer of adhesive being sufficiently soft and pliable to be elastically compressed during use thereby permitting comfortable use of the female external incontinence device.

23. A female external incontinence device as recited in claim 22 wherein said housing has an inner wall surface carrying inwardly extending protuberances whereby when said housing is compressed during use said protuberances prevent said inner wall surface from occluding passage of urine through said housing.

24. A female external incontinence device as recited in claim 22 wherein said wine collection channel and said outlet tube have inner wall surfaces carrying inwardly extending protuberances whereby when said housing is compressed during use said protuberances prevent said inner wall surfaces from occluding passage of urine through said urine collection channel and said outlet tube.

25. A method of using a female external incontinence device comprises the steps of
providing a housing made of soft, pliable, membranous, elastomeric material formed to provide a flange for creating a seal surrounding the parameatal urethral meatus and to provide a urine collection channel;
providing an intravaginal component coupled with the housing;
inserting an applicator carrying the housing and the intravaginal component between the labia to spread the labia;
rotating the applicator to contact the anterior vaginal wall to spread and flatten the rugae in the introitus; and
removing the applicator leaving the housing in place.

26. The method of using a female external incontinence device as recited in claim 26 and further comprising the step of adhesively securing the flange to the intralabial surface to create the seal surrounding the parameatal urethral meatus.
